(19) United States
(12) Patent Application Publication
(10) Pub. No.: US 2022/0148704 A1
(43) Pub. Date: May 12, 2022

(54) NUTRIMENT TRACK AND ALERT SYSTEM

(71) Applicant: Kianoosh Peyvan, Seattle, WA (US)

(72) Inventor: Kianoosh Peyvan, Seattle, WA (US)

(21) Appl. No.: 17/523,891

(22) Filed: Nov. 10, 2021

Related U.S. Application Data
(60) Provisional application No. 63/112,631, filed on Nov. 11, 2020.

(51) Int. Cl. G16H 20/60; G16H 20/10

(52) U.S. Cl. CPC G16H 20/60; G16H 20/10

(57) ABSTRACT

The present disclosure provides a nutriment track and alert system and method. The system includes at least one monitoring station configured to support a container; at least one weight-measuring device for detecting and measuring the container weight and/or weight differentials when a container is placed or lifted on the monitoring station. The monitoring station or weight-measuring device communicates with a computer executable protocol that can be executable on a mobile device, computer, web server, and/or coupled or housed in the system itself. The protocol can store medication data through user input or through communication with the monitoring station or weight-measuring device. When the weight-measuring device or the protocol detects a change in the weight differentials not aligned with the medication data, the system submits and alert.

### BACKGROUND

### Technical Field

The present disclosure generally relates to the field of nutriment or agent (e.g., medication, supplement, suppository, inhalant) consumption adherence, in particular, tools including internet of things (“IoT”) hardware devices and a set of web and mobile services to which the hardware connects through a network protocol.

### Description of the Related Art

Increasingly patients require various medications and supplements, critical to their well-being and/or treatment. The precise dose and timing of medications are critical to these medications being effective and safe for the patient.

There are various tools, gadgets, and methods for organizing and reminding people to take their medications. They fall in a few categories discussed below along with their shortcomings.

1. Pill Organizers

A pill organizers characterized as a pill container, dosette box, pill case or pillbox all basically represent a multicompartment compliance aid for storing scheduled doses of medications. Pill organizers usually have square-shaped compartments for each day of the week, and other more compact and discreet versions have come to market, including cylindrical and pen-shaped cases. Some organizers have multiple sections, corresponding with different times of the day. Pill organizers are viewed as a way to reduce medication errors on the part of the patient. Historically such organizers have not typically been effective given heavy reliance on user memory, and they have been linked to medication errors. See https://en.wikipedia.org/wiki/Pill_organizer.

2. Pill Dispensers

Pill dispensers are devices which release medication at specified times. Their purpose is to help senior citizens and other people who may suffer from impaired ability to adhere to their prescribed medication regime.

Pill dispensers are commonly used to serve medical purposes, as well as to help individuals, be that the elderly or chronically ill, take their prescription medication, over the counter medication, or daily supplements at a given date and time. There are telehealth products in the health care industry such as automated or electronic pill boxes that function to alert the patients when it is time to take their medications.
A. First Generation Automatic Pill Dispensing System

Automatic Pill Dispensing Systems are usually manufactured with a rotating tray containing multiple compartments, where each compartment is intended for a single dosage. These pill reminders are programmable and usually dispense medication according to only the alarm set by the user. Each compartment on the tray is pre-filled at a specific location with the required dosages weekly or monthly. When it is time to take a dose the tray rotates to the dispensing location and the pills can be accessed through an opening, that is the pill is dispensed. Some models could include a flashing light and audible alarm that sounds when a dosage is recommended.
B. Second Generation Pill Dispensing Systems

These devices, although slightly improve over the first-generation dispensers, they are still considered a dispensing device. These units are larger than rotating pill dispensers and have separate internal storage for each prescription. The user fills each compartment with an entire pill bottle content and the instrument is then programmed how to dispense each compartment at a programmed date and time.

3. Smart Phone Pill Reminder Applications

Software companies have developed simple applications available for most smart phone platforms. These applications aim to aid users manage their medication intake in terms of tracking past usage and sending reminders when they are due.

4. Timed Pill Bottle Caps

Specialized bottles with timers built into their caps are also available commercially. The caps for these bottles contain a microcontroller and a switch and a display. The switch can reset a timer indicating the last time the bottle was opened as a surrogate of medication intake. In some version of these products the timer is activated when the user presses a switch. In other embodiment the switch is activated automatically when the bottle is opened or closed. In recent developments these bottle caps can connect to a mobile device via Bluetooth® or other means of communications thus allowing the mobile device or web services to record the time the bottle was opened or closed. Some can also send refill reminders when total open/close events equal total quantity intake prescription as well as submitting refill signals.

5. Related Art Drawbacks

The products discussed above have numerous drawbacks and disadvantages. By way of non-exhaustive examples, they all require the user to remove the medication from its casing, in some cases the child proof casing, and manually load it to a separate custom container. In the case of the pill organizer and the first generation pill dispenser this removal and replacement must occur frequently, for example every day or every week. The more recent sensing cap trackers track open/close events, not detecting whether a dose was removed from the container and require a distinct sensing cap for each prescription. These processes are tedious and error prone, especially for vulnerable patients such as the elderly and those suffering from memory and/or chronic issues impacting their attention and motor skills.

All of these methods also lack a feedback mechanism for the user or the user's caregiver to indicate whether or not the user has taken the medication or supplement.

Moreover, the various container and dispenser products require numerous steps and parts, especially when multiple medications or supplements are involved. These drawbacks also make for expensive products and processes often out of the reach of vulnerable patients. Furthermore, software applications can be difficult to learn, operate, and maintain for such patients.

### BRIEF SUMMARY

According to one embodiment, a nutriment adherence system includes at least one internet of things (IoT) hardware device and web and mobile services to which the device connects or communicates via a network protocol. Together, the device and the services facilitate monitoring a time and amount of a nutriment taken and remind the users, caregivers, or the medical professionals if a patient misses a dose. The reminders can be presented in many forms such as audio, visual indication on the device or on another device, popup notifications on a smart phone, or electronic notifications such as via text or email.

According to one embodiment, a nutriment track and alert system includes at least one monitoring station configured to support a nutriment container containing a plurality of nutriment doses, at least one weight-measuring system having a weight-measuring device in communication with the monitoring station and configured to detect and measure a weight of the nutriment container, and a communication system including an analog to digital converter in electronic communication with the weight-measuring device and operable to convert an analog weight signal to a digital signal, a micro-controller in electronic communication with the analog to digital converter and operable to receive the digital weight signal and communicate the same to at least one of a mobile computing device and a web server.

The system according to one aspect includes a base coupled to the monitoring station and at least one of housing and being coupled to the weight-measuring system, the weight-measuring device including a load cell arrangement having a plurality of strain gauges at least some of which are stressed and some are unstressed.

In one aspect, the communication system further includes a computing device executable protocol configured to at least one of receive and store medication data including at least one of nutriment dosage, consumption interval, and dose quantity information via at least one of user input through the computing device, the protocol, and electronic communication with the weight-measuring system, the protocol in electronic communication with the micro-controller.

According to one aspect, the weight-measuring device is operable and configured to detect a first weight differential upon removal of the nutriment container from the monitoring station and a second weight differential upon replacement of the nutriment container on the monitoring station, at least one of the weight-measuring device and the protocol operable to calculate a difference between the first weight differential and the second weight differential, and submit an alert through the communication system to at least one of the computing device and the web server upon detection and identification of a weight change pattern incongruent with the medication data stored in the protocol.

The system, according to one aspect, includes at least one of an audible signal generator and a visual indication generator, wherein the weight-measuring device is configured and operable to detect a first weight differential upon lifting the nutriment container from the monitoring station and a second weight differential upon replacement of the nutriment container on the monitoring station, at least one of the weight-measuring device and the protocol configured and operable to calculate a difference between the first weight differential and the second weight differential, at least one of the analog to digital converter and microcontroller communicating with the at least one of the audible signal generator to generate an audible alert and the visual indication generator to generate a visual alert, upon detection and identification of the difference between the first and second weight differentials being incongruent with the medication data.

According to another embodiment, a method for tracking nutriment consumption includes monitoring a weight of a nutriment container containing a plurality of nutriment doses using a weight-measuring device, measuring and identifying a first weight differential upon lifting the nutriment container from the weight-measuring device and a second weight differential upon replacement of the nutriment container on the weight-measuring device, calculating a weight difference between the first weight differential and the second weight differential, storing medication data including nutriment dosage, consumption interval, and dose quantity information in a computing device executable protocol via at least one of user input through a computing device and electronic communication with the weight-measuring device, converting analog weight signals to digital signals, and communicating the digital signals with at least one of the computing device and a web server.

In one aspect, the method includes submitting an alert to at least one of the computing device and the web server upon detection and identification of the difference between the first and second weight differentials being incongruent with the medication data stored in the protocol.

In one aspect, the method further includes generating at least one of an audible alert and a visual indication upon detection and identification of the difference between the first and second weight differentials being incongruent with the medication data stored in the protocol.

### BRIEF DESCRIPTION OF THE SEVERAL VIEWS OF THE DRAWINGS

FIG. 1 illustrates an isometric view of a nutriment track and alert system according to one embodiment.

FIG. 2 illustrates an isometric view of the nutriment track and alert system of FIG. 1 with two nutriment containers positioned on respective monitoring stations thereof, according to one embodiment.

FIG. 3 is a schematic front view of a computing device displaying example metrics of a computing device executable application or protocol operable to be in digital, electronic, and/or wireless communication with the nutriment track and alert system of FIG. 1, according to one embodiment.

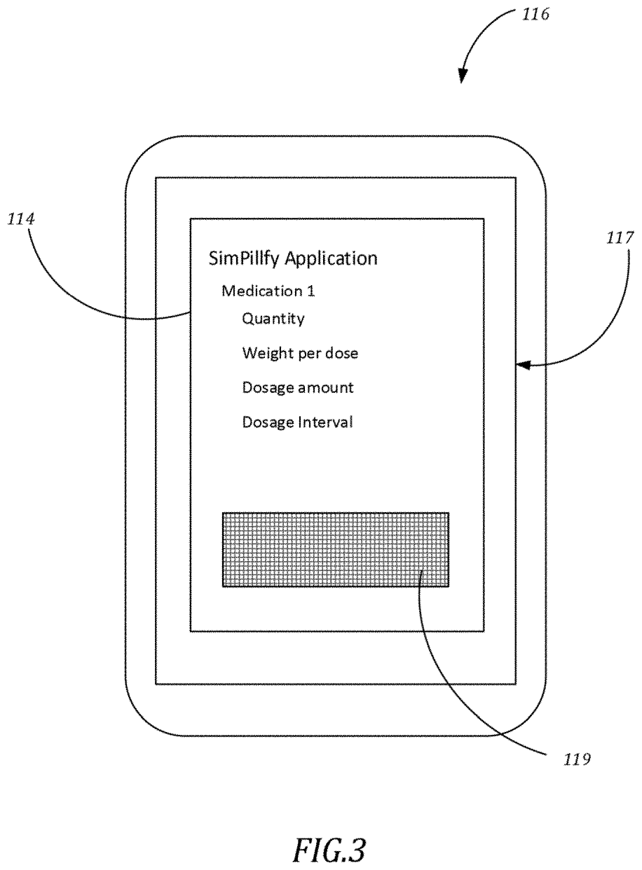

FIG. 4 illustrates a bottom view of the nutriment track and alert system of FIG. 1, according to one aspect.

FIG. 5 illustrates a close up of a portion of a communication system of FIG. 6, according to one embodiment.

FIG. 6 illustrates a schematic of a nutriment track and alert communication system according to one embodiment.

FIG. 7 illustrates a schematic of a portion of the communication system of a nutriment track and alert system according to one embodiment.

FIG. 8 illustrates a side view of the nutriment track and alert system of FIG. 1, according to one aspect.

FIG. 9 illustrates a top view of the nutriment track and alert system of FIG. 1, according to one aspect.

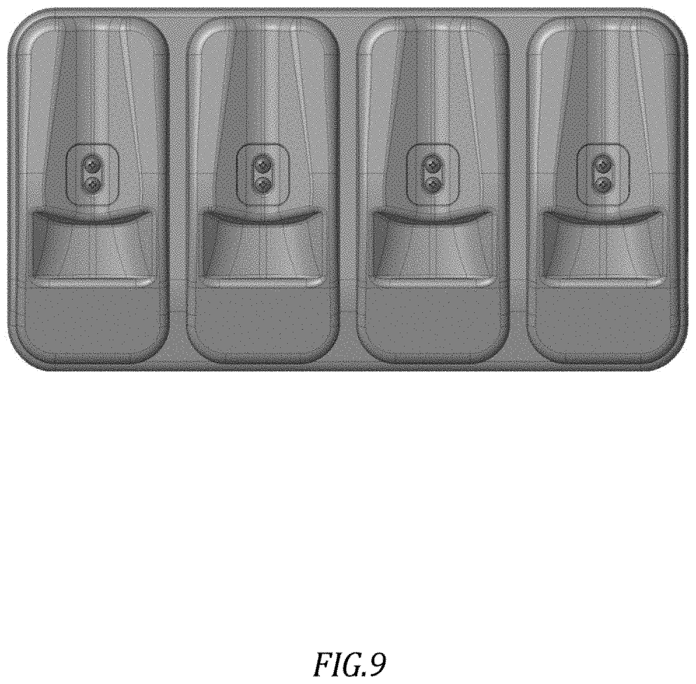

FIG. 10 is a flow diagram that illustrates a process for tracking nutriment consumption.

FIG. 11 is a block diagram of an example computing system or device for implementing a nutriment tracking system or element thereof according to an example embodiment.

### DETAILED DESCRIPTION

In this disclosure various example embodiments of a nutriment track and alert system are provided. Solely for clarity of language and without any intention to limit the scope, “nutriment track and alert system” in certain places is referred to as “nutriment track system” or “medication track system” and “nutriment” in certain places is referred to as “medication.” It is understood that nutriment and/or medication in this disclosure encompasses any and all agents a user or patient may consume for any reason such as for treatment, health, well-being, energy, sleep, nutrition, etc. Such nutriments and/or medications can include, among others, prescribed or over the counter drugs and medications, supplements, vitamins, protein agents, suppositories, inhalants, reagents, chewable agents, intradermal delivery devices, etc.

As used herein, the term “user” can include a patient or any other interested person such as an individual intending to consume nutriments for any reason, and “caregiver” can include any other interested individual including without limitation family members, healthcare professionals, employees, and/or insurance representatives.

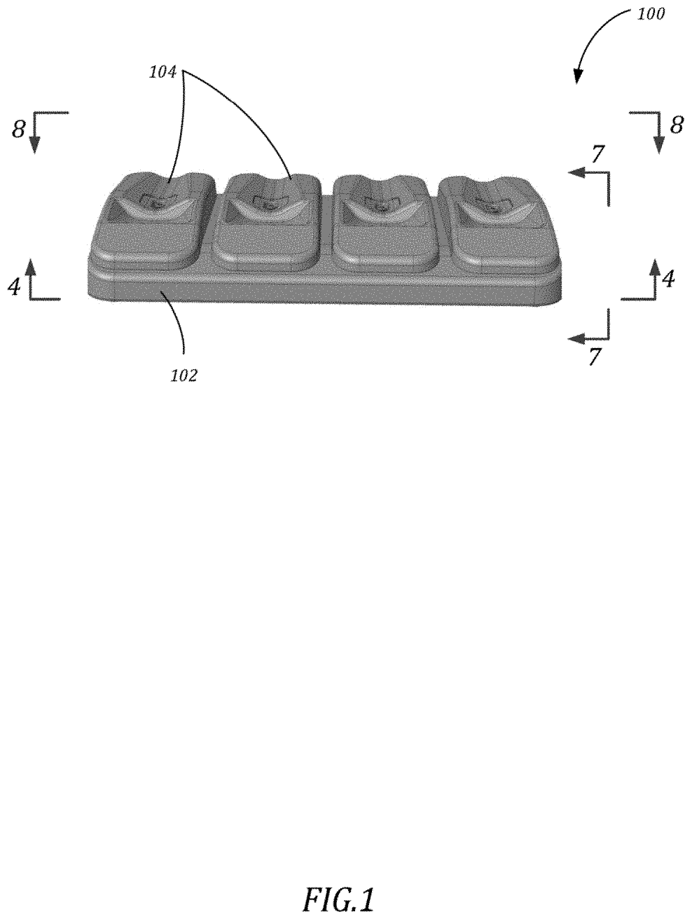

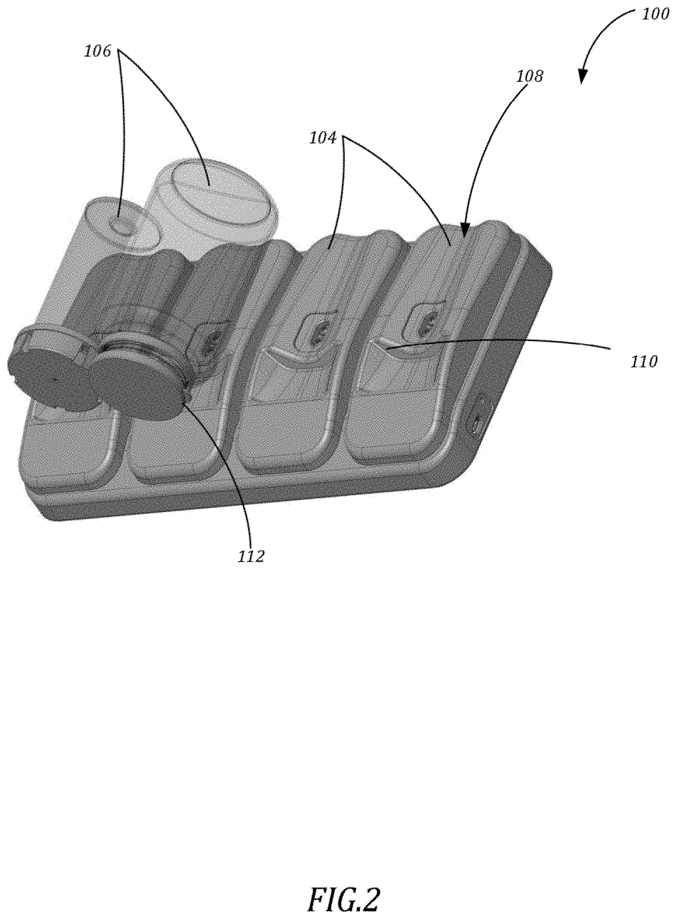

According to one embodiment illustrated in FIG. 1, a nutriment track system 100 includes a base or platform 102 and at least one monitoring station 104. As illustrated in FIG. 2, in on aspect, the monitoring station 104 is configured to at least partially support or receive a nutriment container 106, which in turn can hold or contain medication doses. The monitoring stations 104 can each include any shape or contour designed to receive nutriment containers 106 of any shape or contour. For example, in one aspect, the monitoring stations 104 can include a concave contour 108 shaped complementary to an outer surface of the corresponding nutriment container 106. In some embodiments the monitoring station 104 can include a lip or protrusion 110 configured to be positioned adjacent or contiguous a lid portion 112 of the nutriment container 106 to further secure the nutriment container 106 in place.

In one embodiment, the monitoring stations 104 are or can operate independent one another. According to one aspect, a user such as a patient under treatment, places the nutriment container 106 on one of the monitoring stations 104.

In one embodiment, the system 100 includes a computing device executable protocol or application 114 configured to receive user input information. In one aspect, the application 114 is executable on a computing device 116, such as a laptop having a user readable screen or monitor 117 and control panel 119 configured to receive user input for example alphanumeric values. The computing device 116 can include a mobile device such as a smart mobile phone, laptop, workstation, tablet, dedicated computing device, and/or any other suitable computing device, for example internet and/or web application executable platforms. In some embodiments, a computing device can be coupled to or integrated with the base or platform 102 or one or more monitoring stations 104.

In one aspect, the application 114 is configured to connectedly, wirelessly, electronically and/or digitally communicate with the monitoring station(s)104 as further described with respect to some embodiments.

In one embodiment, upon installing the application 114 on the computing device 116, the user can create and thereafter log in using credentials such as a username and a password. In one aspect, once the application 114 is accessed, the user can input information via the control panel 119 relating to each medication corresponding to respective monitoring station(s) 104, which information will be visible to the user on the monitor 117. For example, the user can input medication dosage quantity, weight, and consumption interval (“Medication Data”) for each medication. In one aspect, the nutriment track and alert system 100 or a portion thereof is configured to measure and/or identify Medication Data and communicate the same to protocol 114 programing to be stored for respective nutriments.

For example, the protocol programming may include information by prescription identification and/or to be in wireless, digital, and/or electronic communication with the system 100 and its weight-measuring and communication features, disclosed below for some embodiments, and receive Medication Data such as weight and quantity by the user placing one dose on the monitoring station 104, the monitoring station 104 measuring and communication the Medication Data to the protocol 114 to be visible by the user and to store in the protocol 114 for respective nutriments. In some embodiments, the monitoring station 104 and/or its weight-measuring features and/or the protocol 114 can be configured or programmed to detect the weight of the medication container 106 (FIG. 2) and subtract it from the calculation. In some embodiments, the monitoring station 104 can include or be coupled to a scanner to scan prescription information from the container 106 and convey corresponding Medication Data to the protocol 114. In some embodiments, the protocol 114 can include scanning features to scan barcodes or information on the container 106 (FIG. 2) and auto-fill Medication Data in the protocol.

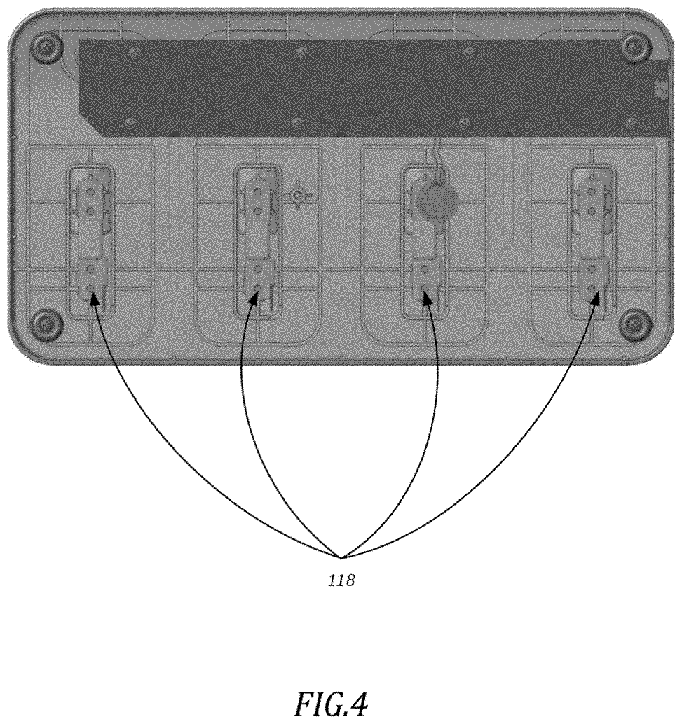

In one embodiment, the system 100 and/or the respective monitoring stations 104 include a weight-measuring system 118, generally illustrated in FIG. 4 according to one example embodiment.

In application in one aspect, the user positions the medication container 106 on one of the monitoring stations 104, and inputs the Medication Data in the application 114 for medication contained in the medication container 106. In one embodiment, the user can initiate a start of a consumption interval using the application 114 commands. In one embodiment, placement of the medication container 106 on the monitoring station 114 can initiate the start of the consumption interval. In operation in one embodiment, when the user lifts up the container 106, the monitoring station 104 detects a dosage consumption event. Upon the user removing and repositioning the medication container 106 on the monitoring station 104, the monitoring station 104 is configured and/or equipped to detect whether or not a medication dosage was subtracted from the medication container 106 by calculating a weight of the medication container 106 via the weight-measuring system 118 following repositioning of the container 106 and comparing the same to the weight change experienced by the weigh-measuring system 118 when the user picked up the medication container 106.

If the medication container 106 weight is substantially unchanged, the monitoring station 104 can submit an alert to the user and/or caregiver to alert that the dosage may not have been consumed. If monitoring station 104 detects the weight of the medication container 106 to be less by the weight of one dosage as entered in the application 114, the monitoring station 104 can restart the consumption interval calculation. In one aspect, if the monitoring station 104 detects a weight reduction heavier than one dosage and/or a consumption event occurring earlier than the scheduled interval stored in the application 114, the monitoring station 104 can submit an alert to the user or caregiver.

In one aspect, if the monitoring station 104 does not detect a weight change corresponding to the prescribed consumption event according to the interval schedule in the application 114, the monitoring station can submit an alert to warn the user or caregiver with a reminder that a dosage is due for consumption.

The following disclosure is related to one embodiment of operation of the monitoring station in communication with the application 114 according to a weight-monitoring system and method, as one example of weight monitoring and communication protocol to effect the above track and alert functions.

In various embodiments, the respective monitoring stations 104 can include any suitable weight-measuring system, which can be the same as one another or distinct from one another. In one embodiment, as illustrated in FIGS. 4, 5, and 6, the weight-measuring system 118 includes a load cell arrangement 120 including two or more, or a plurality of strain gauges. The weight-measuring system(s) 118 can in one aspect be coupled and or in communication with the respective monitoring stations 104 to detect and/or measure a weight in response to pressure differentials on the monitoring stations. For example, in one aspect, each weight-measuring system 118 can be integrated or contained at least partially in the base 102 and/or the respective corresponding monitoring stations 104, or coupled thereto.

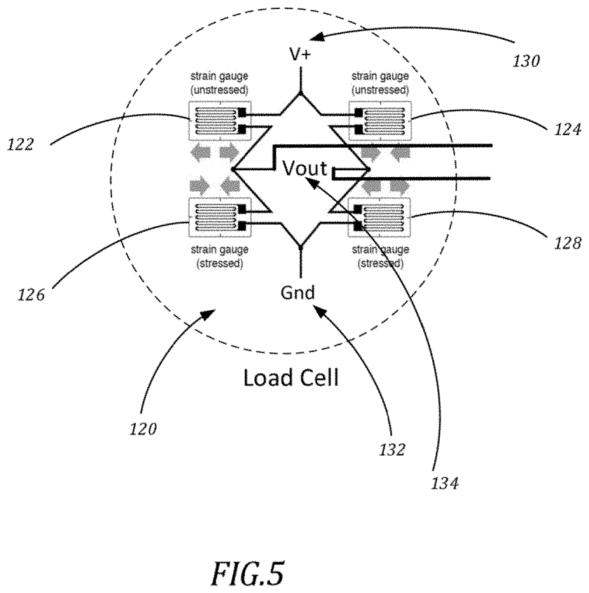

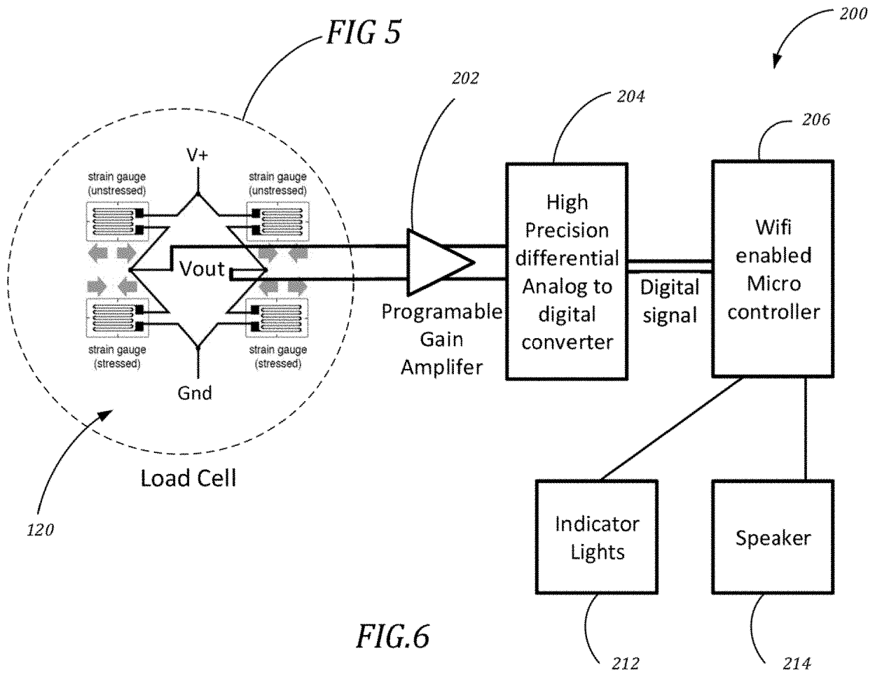

For example, as illustrated in FIGS. 5 and 6, in one embodiment, the load cell arrangement 120 can include first and second opposing unstressed strain gauges 122, 124, first and second opposing stressed strain gauges 126, 128, at least one power source 130 in electronic communication with the strain gauges 122, 124, 126, 128, a ground 132, and an electronic out communication element or voltage out 134.

The power source 130 supplies voltage or electric power to the load cell arrangement 120, can include power transfer from any suitable power source such as, but not limited to, a lithium ion battery that in various embodiments can be integrated with the monitoring system 104 and/or load cell 120, or external thereto. Other power sources are contemplated to be with the scope of the present disclosure.

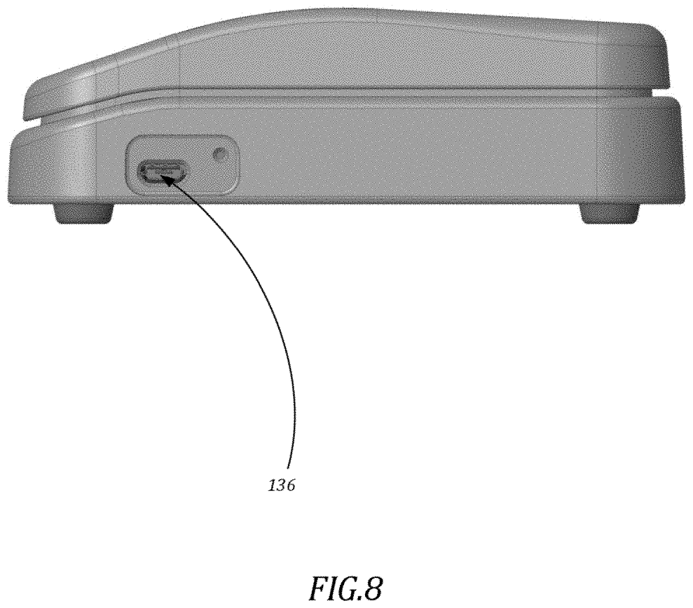

In one aspect, the nutriment track and alert system 100 can be powered by a power source, such as, but not limited to, a lithium, alkaline, and zinc batteries. Examples can include alkaline cell batteries, cylindrical batters such as AA or AAA batteries, and various lithium ion batteries. The power source in various embodiments can be integrated with the monitoring system 104 or external thereto. In some embodiments, the power source can include devices to draw electrical power from an outlet or port. For example, as illustrated in FIG. 8, the system 100 can incorporate a Universal Serial Bus (USB) port 136 configured to receive a USB chord, or other industry buses and chords, which in turn can be connected to an electronic outlet such as home wall outlets. Other power sources and batteries are contemplated to be within the scope of the present disclosure, such as, but not limited to, silver-oxide batteries. In some aspects, the load cell arrangement 120 can be powered through circuitry electrically in communication with the power source for the nutriment track and alert system 100.

The following disclosure relates to certain example features for facilitating the above-mentioned communication according to one embodiment without intention to limit the scope of the present disclosure.

FIG. 6 illustrates schematics of one embodiment of a nutriment track measurement and communication architecture, and visual and audible alert features that can be integrated or positioned on the system 100, for example coupled to the platform 102 or one or more of the monitoring stations 104, and/or operatively coupled thereto. In one aspect, the nutriment track and alert system 200 includes a communication system 200 having a programable gain amplifier 202, an analog to digital converter 204 such as a high precision differential analog to digital converter, a microcontroller 206 such as a WiFi enabled microprocessor or microcontroller. In one embodiment communication system 200 can include an audible device such as a speaker 214 or other device and/or method of generating an audible signal or play a pre-recorded message. In one embodiment the communication system 200 can include a visual indicator such as indicator lights 212 for example, as LEDs or displays to alert and inform the user and/or caretaker for an action for example when a nutriment is due or that less or more than a dose has been consumed.

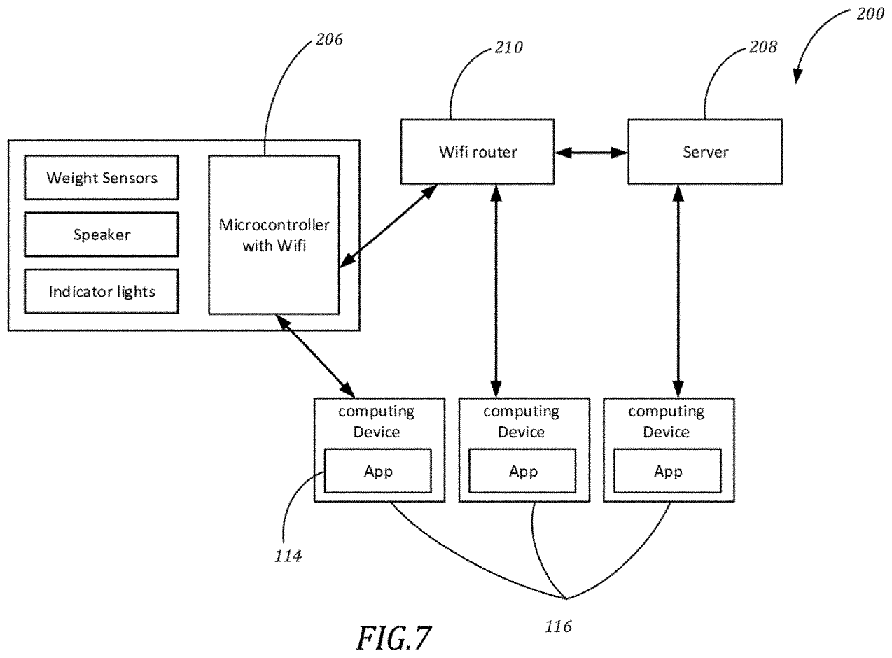

Regarding triggering the alerts, FIG. 7 illustrates a schematic of the nutriment track and alert communication system 200 according to one embodiment incorporating wireless, digital, and/or electronic communication devices and protocols. Referring to FIGS. 2, 6, and 7, in one aspect, the application protocol 114 is executable by the computing device 116, and a remote server 208 such as a web server. In one embodiment, the computing device 116 can be configured to wirelessly, digitally, via Bluetooth® and/or electronically communicate back and forth with the remote server 208, for example via a gateway device such as wireless hub or router 210. In one embodiment the computing device 116 can directly communicate with nutriment track and alert system 100 via any one or more of the above-mentioned communication protocols or standards, bypassing the server 208 through the router 210. Furthermore, the router 210 can be configured to act in certain modes, such as modes referred to as an access point and to allow the computing device 116 to directly access the router 210 and provide a bridge to server 208.

The communication system 200 can enable the monitoring stations 104 to submit the user's nutriment consumption data to the remote server 208, which in turn can submit data and alerts to one or more computing devices 116, for example, mobile devices and/or smart phones belonging to the user and the caregiver(s). In some embodiments, the data and/or alerts can be read from other gateway devices, for example, such as smart scales, vitals monitors, for example using a wireless and/or Bluetooth® connections.

In one embodiment, the load cell 120 conveys the medication container 106 weight with analog signals to the analog to digital converter 204, which digitizes the weight data and feeds the digital weight data to the microcontroller 206. The microcontroller 206 monitors these values and converts them to weight values. The weight values and their change or variance can in one aspect be continuously monitored by the microcontroller 206. In certain cases, the weight values might exhibit slight variations or drift over time due to load cell strain gauge or sensor noise characteristics. However, if the variations over a short time frame for example 1 second are high enough to pass a selected or programmed or selected threshold, the communication system would indicate a weight change event.

The communication system 200 can be configured to detect a weight change event when a weight change pattern defined as all of the measurements observed over a time period while a weight signal was measured is above a predefined noise floor.

These weight change patterns over time can indicated a number of possibilities. For example, if the user accidentally touches the sensor but does not lift the medication container off the monitoring station 104, the signal would revert to the state before the accidental contact and thus treated as a net zero event.

The pattern indicating a user removal and replacement of the medication container 106, would exhibit a net negative and net positive event, respectfully, which would indicate that a bottle may have been lifted off the monitoring station 104 (“Lift Event”) or placed back onto the monitoring station 104 (“Placement Event”). These two events have a distinct pattern which approximates a sigmoidal curve. The curve transitioning from lower to higher ends indicates a medication container 106 Placement Event, while the opposite indicates a medication container 106 Lift Event with respect to the monitoring station 104.

In one aspect, by analyzing the weight change pattern, the system 200 approximates if the user has lifted or placed the medication container 106 on the corresponding monitoring station 104. In one aspect, the system 200 is configured to detect or measure a net negative weight experienced during a Lift Event and compare the same to the weight increase upon a Placement Event. These net weight differentials having been communicated to the mobile computing device 116, the application 114 and/or weight-measuring device 118 can be configured and/or operable to measure the difference between them.

Given the medication single dose weight is known from the single dose weight the user or caregiver has input in the application 114 or utilized the application 114 in conjunction with the monitoring station 104 or its load cell arrangement 120 to measure the weight or other Medication Data during the prescription setup, the application 114 is operable to estimate whether a dose was removed from the medication container 106.

For example if one dose weighs 1 gram and the system 200 measures a net weight at Lift Event of −10 grams followed by a measuring a net weight at Placement Event of +9 grams, the system 200 calculates a net −1 grams or less one dose. All the computation, storage, pattern forming, comparing, and analysis can be performed on the monitoring station 104 or partially on the monitoring station 104 and partially on at least one of the remote server 208 and computing device 116.

On the remote server 208 and/or on the application 114, each dosage change is tagged with a time stamp at which data was received at the server 208 or computing device 116. This time stamp indicates when the dosage was consumed or added.

The time and the dosage consumed is then compared against the prescription or Medication Data entered for the medication corresponding to that monitoring station 104 on the server 208 software and/or the application 114. The server 208 and/or the application 114 calculates the next time a dosage should be taken and restarts the interval timer based on the user input interval entered in the application 114. If the server receives proper event indicating the user has taken the right amount and quantity of the dosage at a specified time then it resets the timer for the next time the user needs to take a dose. If the server did not receive the correct dosage change event at the corresponding time, it will notify the user and/or caregiver that the prescription intake is due or overdue, or potentially consumed at a higher or lower dose than that prescribed.

In various embodiments, the notifications can occur in one or more manners. In various aspects, the notification can be displayed on the computing device 116, sent via text message or email to the user or caregiver, sent from the server 208 to the corresponding monitoring station 104, triggering that monitoring station 104 to display a visual indication such as light flashing via indicator lights 212 at the that station 104, or playing a sound or a prerecorded audio via speaker 214.

It is understood that a nutriment track and alert system according to various embodiments can be configured to leverage an aspect of a weight-measuring and communication system and protocol to achieve stated and unstated functions or goals.

For example, in one embodiment, the application 114 can receive a preferred pharmacy information and pharmacy systems can serve as a gateway or web server. In such applications, for each medication container 106 the corresponding monitoring station 104 having with the weight-measuring system 118 or other suitable weight-measuring system can detect when all the medication therein has been consumed based on the quantity and weight of each dose entered in the application 114 and communicated to the corresponding monitoring station 104. The monitoring station 104 and/or the application 114 can be programmed or configured to communicate through a communication as stated above or similar communication protocol to the user's or caregiver's computing device 116 and/or to the pharmacy system to automatically trigger a refill order if one is available in the corresponding prescription.

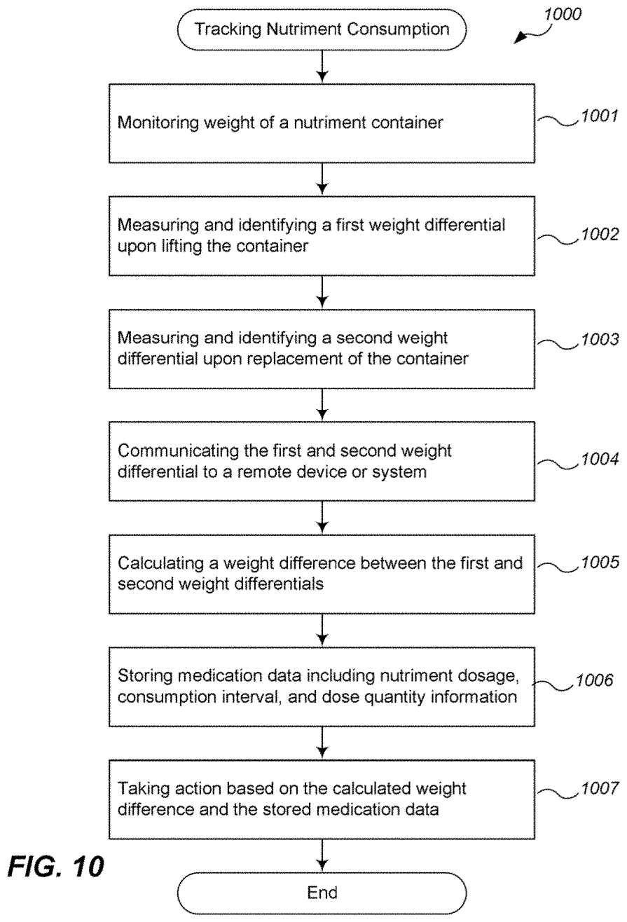

FIG. 10 is a flow diagram that illustrates a process 1000 for tracking nutriment consumption. This process may be performed by a microcontroller or similar in the system 100 or 200 or a combination of the two. Some steps of the process may also or instead be performed by the computing device 116 and/or server 208.

Block 1001 includes monitoring a weight of a nutriment container containing a plurality of nutriment doses using a weight-measuring device.

Block 1002 includes measuring and identifying a first weight differential upon lifting the nutriment container from the weight-measuring device. In some embodiments, the process detects a lift event by monitoring a load cell or similar hardware and determining whether the load value decreases more than a selected or programmed threshold over a certain time period (e.g., 1 second, 2 seconds, 5 seconds). In other embodiments, other or additional mechanisms or hardware may be used to measure the weight of the nutriment container, such as a strain gage, capacitive sensor, or the like.

Block 1003 includes measuring and identifying a second weight differential upon replacement of the nutriment container on the weight-measuring device. The process may detect a placement event by monitoring a load cell and determining whether the load value output by the load cell increases by more than a threshold amount over a specified time period.

Block 1004 includes communicating the first and second weight differential to a remote device or system. To communicate the weight, the process may first convert analog weight signals to digital signals, and then communicate the digital signals with at least one of the computing device and a web server. In some embodiments, the process may also or instead periodically take and transmit weight measurements that may or may not be triggered by lifting and replacement of the container. For example, the process may transmit a stream of weight values sampled every N seconds (independent of whether the container is being lifted or replaced upon the weight measurement device), such that the remote system can determine whether the container has been lifted, replaced, or otherwise based on patterns in the received weights. The remote system may identify such patterns in some embodiments by use of machine learning (e.g., logistic regression, decision trees, neural networks).

Block 1005 includes calculating a weight difference between the first weight differential and the second weight differential. Typically, this operation is performed on the server 208. In other embodiments, this calculation can be performed on the device 116 or even on the system 100.

Block 1006 includes storing medication data including nutriment dosage, consumption interval, and dose quantity information. The data may be stored in a computing device or a server executable protocol via at least one of user input through a computing device and electronic communication with the weight-measuring device. Typically, this operation is performed on the server 208. In other embodiments, this calculation can be performed on the device 116 or even on the system 100.

Block 1007 includes taking action based on the calculated weight difference and the stored medication data. For example, the process may compare the weight difference against the recommended dosing regimen, and then send a notification based on that comparison, such as an alert or reminder to take or adjust a dose.

In typical embodiments, blocks 1001-1004 are performed by a microcontroller or similar processor resident in the system 100, while blocks 1005-1007 are performed by the device 116 (e.g., smart phone running an app) or the server 208. In other embodiments, one or more steps or operations may be performed by other devices or systems and/or in different orders.

The described techniques and components may be employed in other form factors in other embodiments. For example, in one embodiment a weight measuring device and communication subsystem may be housed in a bottle cap. This bottle cap can then be attached to a pill container or similar. The container is then stored resting on the cap, such that the cap can measure and communicate the contents of the container. In another embodiment, the weight measuring device and communication subsystem are housed in container. The user transfers their pills or other nutriments to the container (or places their pill bottle within the container), which can then measure and communicate the weight of the pills.

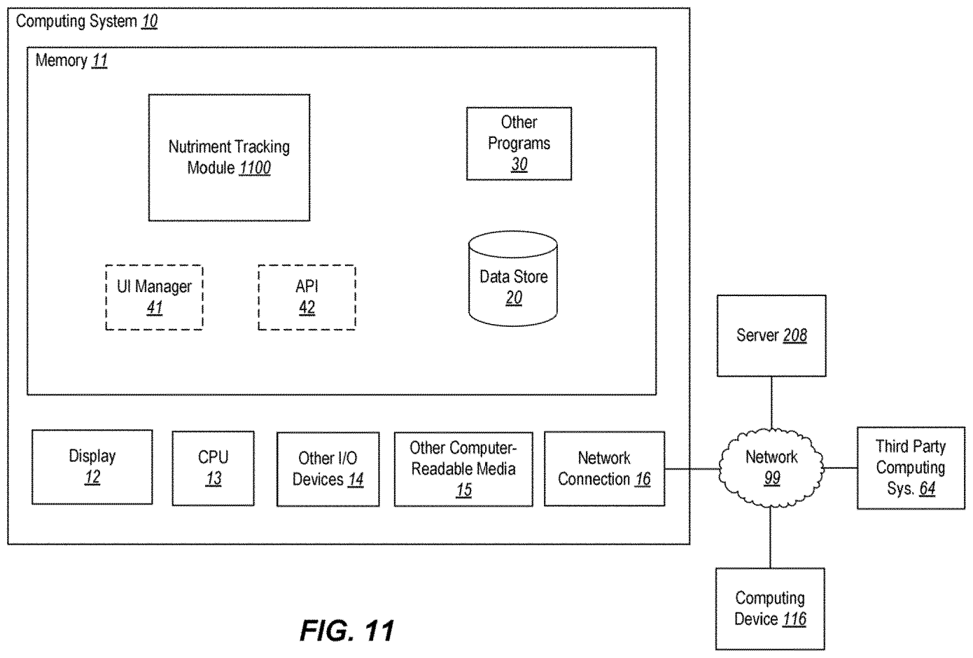

FIG. 11 is a block diagram of an example computing system or device for implementing a nutriment tracking system or element thereof according to an example embodiment. In particular, FIG. 11 shows a computing system 10 that executes a module 1100 that implements at least some of the techniques described herein. The computing system may be, for example, the microcontroller 206 discussed above.

In the embodiment shown, computing system 10 comprises a computer memory (“memory”) 11, a display 12, one or more Central Processing Units (“CPU”) 13, Input/Output devices 14 (e.g., keyboard, mouse, CRT or LCD display, and the like), other computer-readable media 15, and a network connection 16. The module 1100 is shown residing in memory 11. In other embodiments, some portion of the contents, some or all of the components of the module 1100 may be stored on and/or transmitted over the other computer-readable media 15. The module 1100 preferably executes on one or more CPUs 13 and performs the techniques described herein. Other code or programs 30 (e.g., an administrative interface, a Web server, and the like) and potentially other data repositories, such as data repository 20, also reside in the memory 11, and preferably execute on one or more CPUs 13. Of note, one or more of the components in FIG. 11 may not be present in any specific implementation. For example, some embodiments may not provide other computer readable media 15 or a display 12.

The module 1100 is shown executing in the memory 11 of the device 100. Also included in the memory 11 are a user interface manager 41 and an application program interface (“API”) 42. The user interface manager 41 and the API 42 are drawn in dashed lines to indicate that in other embodiments, functions performed by one or more of these components may be performed externally to the module 1100.

In typical movements, most if not all user interface functions are performed by a remote mobile device (e.g., smart phone) or similar. However, some embodiments may include a user interface manager 41 that is responsible for managing any user-related interactions, such as audible or visual notifications (e.g., beeps, warning chimes, blinking lights) and/or inputs (e.g., a reset button).

The API 42 provides programmatic access to one or more functions of the module 1100. For example, the API 42 may provide a programmatic interface to one or more functions of the module 1100 that may be invoked by one of the other programs 30 or some other module. In this manner, the API 42 facilitates the development of third-party software, such as user interfaces, plug-ins, adapters (e.g., for integrating functions of the module 1100 into Web applications), and the like.

The module 1100 may interact using network connection 16 via a network 99 with other devices/systems including computing systems 64, 116, and 208. The network 99 may be any combination of media (e.g., twisted pair, coaxial, fiber optic, radio frequency), hardware (e.g., routers, switches, repeaters, transceivers), and protocols (e.g., TCP/IP, UDP, Ethernet, Wi-Fi, WiMAX) that facilitate communication between remotely situated humans and/or devices.

Note that one or more general purpose or special purpose computing systems/devices may be used to implement and/or execute the module 1100. However, just because it is possible to implement the module 1100 on a general purpose computing system does not mean that the techniques themselves or the operations (taken alone or in combination) required to implement the techniques are conventional or well known. The techniques are not conventional at least because they address and improve an existing technology, such as by improving the operation, integration, or efficiency of one or more computing systems.

In an example embodiment, components/modules of the module 1100 are implemented using software programming techniques. For example, the module 1100 may be implemented as a “native” executable running on the CPU 13, along with one or more static or dynamic libraries. In other embodiments, the module 1100 may be implemented as instructions processed by a virtual machine that executes as one of the other programs 30.

The various components may be implemented using more monolithic programming techniques, for example, as an executable running on a single CPU computer system, or alternatively decomposed using a variety of structuring techniques, including but not limited to, multiprogramming, multithreading, client-server, or peer-to-peer, running on one or more computer systems each having one or more CPUs. Some embodiments may execute concurrently and asynchronously, and communicate using message passing, remote procedure call, or other distributed computing paradigms. Equivalent synchronous embodiments are also supported. Also, other functions could be implemented and/or performed by each component/module, and in different orders, and by different components/modules, yet still achieve the described functions.

In addition, programming interfaces to the data stored as part of the module 1100, such as in the data store 20, can be available by language-specific APIs; libraries for accessing files, databases, or other data repositories; through representational languages such as XML; or through Web servers, FTP servers, or other types of servers providing access to stored data. The data store 20 may be implemented as one or more database systems, file systems, or any other technique for storing such information, or any combination of the above, including implementations using distributed computing techniques.

Furthermore, in some embodiments, some or all of the components of the module 1100 may be implemented or provided in other manners, such as at least partially in firmware and/or hardware, including, but not limited to one or more application-specific integrated circuits (“ASICs”), standard integrated circuits, controllers executing appropriate instructions, and including microcontrollers and/or embedded controllers, field-programmable gate arrays (“FPGAs”), complex programmable logic devices (“CPLDs”), and the like. Some or all of the system components and/or data structures may also be stored as contents (e.g., as executable or other machine-readable software instructions or structured data) on a computer-readable medium (e.g., as a hard disk; a memory; a computer network or cellular wireless network or other data transmission medium; or a portable media article to be read by an appropriate drive or via an appropriate connection, such as a DVD or flash memory device) so as to enable or configure the computer-readable medium and/or one or more associated computing systems or devices to execute or otherwise use or provide the contents to perform at least some of the described techniques. Some or all of the components and/or data structures may be stored on tangible, non-transitory storage mediums. Some or all of the system components and data structures may also be stored as data signals (e.g., by being encoded as part of a carrier wave or included as part of an analog or digital propagated signal) on a variety of computer-readable transmission mediums, which are then transmitted, including across wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of this disclosure may be practiced with other computer system configurations.

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

While embodiments of the invention have been illustrated and described, as noted above, many changes can be made without departing from the spirit and scope of the invention. Aspects of the embodiments can be modified, if necessary, to employ concepts of the various patents, applications and publications to provide yet further embodiments. The various embodiments described above can be combined to provide further embodiments. Accordingly, the scope of the invention is not limited by the above disclosure.

1. A nutriment track and alert system comprising:
at least one monitoring station configured to support a nutriment container containing a plurality of nutriment doses;
at least one weight-measuring system having a weight-measuring device in communication with the monitoring station and configured to detect and measure a weight of the nutriment container; and
a communication system including an analog to digital converter in electronic communication with the weight-measuring device and operable to convert an analog weight signal to a digital signal, a micro-controller in electronic communication with the analog to digital converter and operable to receive the digital weight signal and communicate the same to at least one of a mobile computing device and a web server.

2. The system of claim 1, further comprising:
a base coupled to the monitoring station and at least one of housing and being coupled to the weight-measuring system, the weight-measuring device including a load cell arrangement having a plurality of strain gauges at least some of which are stressed and some are unstressed.

3. The system of claim 1 wherein the communication system further includes:
a computing device executable protocol configured to at least one of receive and store medication data including at least one of nutriment dosage, consumption interval, and dose quantity information via at least one of user input through the computing device, the protocol, and electronic communication with the weight-measuring system, the protocol in electronic communication with the micro-controller.

4. The system of claim 3 wherein the weight-measuring device is operable and configured to detect a first weight differential upon removal of the nutriment container from the monitoring station and a second weight differential upon replacement of the nutriment container on the monitoring station, at least one of the weight-measuring device and the protocol operable to calculate a difference between the first weight differential and the second weight differential, and submit an alert through the communication system to at least one of the computing device and the web server upon detection and identification of a weight change pattern incongruent with the medication data stored in the protocol.

5. The system of claim 3, further comprising:
at least one of an audible signal generator and a visual indication generator, wherein the weight-measuring device is configured and operable to detect a first weight differential upon lifting the nutriment container from the monitoring station and a second weight differential upon replacement of the nutriment container on the monitoring station, at least one of the weight-measuring device and the protocol configured and operable to calculate a difference between the first weight differential and the second weight differential, at least one of the analog to digital converter and microcontroller communicating with the at least one of the audible signal generator to generate an audible alert and the visual indication generator to generate a visual alert, upon detection and identification of the difference between the first and second weight differentials being incongruent with the medication data.

6. A nutriment track and alert system comprising:
a first device that includes a computer processor and a weight measuring device, wherein the first device is configured to:
monitor a weight of a nutriment container containing a plurality of nutriment doses using a weight-measuring device;
measure and identifying a first weight differential upon lifting the nutriment container from the weight-measuring device and a second weight differential upon replacement of the nutriment container on the weight-measuring device; and
communicate the first and second weight differential to a remote computing system or device;
a second device that includes a computer processor and that is remote from the first device, wherein the second device is configured to:
receive the first and second weight differential from the first device;
calculate a weight difference between the first weight differential and the second weight differential;
store medication data including nutriment dosage, consumption interval, and dose quantity information in a computing device executable protocol; and
take an action based on the calculated weight difference and the stored medication data.

7. The system of claim 6, wherein the second device takes an action by:
submitting an alert to at least one of the first device, a mobile computing device, or a web server upon detection and identification of the difference between the first and second weight differentials being incongruent with the medication data stored in the protocol.

8. The system of claim 6, wherein the second device takes an action by:
generating at least one of an audible alert and a visual indication upon detection and identification of the difference between the first and second weight differentials being incongruent with the medication data stored in the protocol.

9. The system of claim 6, wherein the second device is a mobile computing device.

10. The system of claim 6, wherein the second device is a server computing system executing a Web server.

11. A computer-readable medium that stores instructions that are configured, when executed by a computer processor, to perform a method comprising:
monitoring a weight of a nutriment container containing a plurality of nutriment doses using a weight-measuring device;
measuring and identifying a first weight differential upon lifting the nutriment container from the weight-measuring device and a second weight differential upon replacement of the nutriment container on the weight-measuring device; and
communicating the first and second weight differentials to a remote computing device that is configured to:
calculate a weight difference between the first weight differential and the second weight differential;
store medication data including one or more of nutriment dosage, consumption interval, and dose quantity information in a computing device executable protocol via at least one of user input through a computing device and electronic communication with the weight-measuring device; and
transmit a notification based on the calculated weight difference and the stored medication data.

12. The computer-readable medium of claim 11, wherein the remote computing device is configured to:
submit an alert to at least one of the computing device and the web server upon detection and identification of the difference between the first and second weight differentials being incongruent with the medication data stored in the protocol.

13. The computer-readable medium of claim 11, wherein the remote computing device is configured to:
generate at least one of an audible alert and a visual indication upon detection and identification of the difference between the first and second weight differentials being incongruent with the medication data stored in the protocol.

14. The computer-readable medium of claim 11, wherein the remote computing device is a mobile device that communicates wirelessly with the computer processor.
